(19) United States
(12) Patent Application Publication
(10) Pub. No.: US 2023/0317261 A1
(43) Pub. Date: Oct. 5, 2023

(54) AUTOMATED REGULATORY DECISION-MAKING FOR COMPLIANCE

(71) Applicant: IQVIA Inc., PARSIPPANY, NJ (US)

(72) Inventors: Olaf Vanggaard, Parsippany, NJ (US); Olatz Fruniz, Parsippany, NJ (US); Barry Ahrens, Fayetteville, NC (US); Melanie Brewer, Santa Barbara, CA (US); Gary Shorter, Cary, NC (US)

(21) Appl. No.: 18/131,256

(22) Filed: Apr. 5, 2023

Related U.S. Application Data
(60) Provisional application No. 63/327,571, filed on Apr. 5, 2022.

(51) Int. Cl. G16H 40/20; G06Q 30/018

(52) U.S. Cl. CPC G16H 40/20; G06Q 30/018

(57) ABSTRACT

A computer-implemented method includes receiving, by a machine learning model, a question associated with healthcare compliance from a user; identifying, by the machine learning model, a healthcare compliance regulation document associated with the question and one or more healthcare compliance requirements corresponding to the healthcare compliance regulation document; and recommending, by the machine learning model, a decision satisfying the one or more healthcare compliance requirements to the user.

### CROSS-REFERENCE TO RELATED APPLICATIONS

This application claims the benefits under 35 USC § 119(e) of U.S. Provisional Patent Application No. 63/327,571, entitled “Regulatory Management System,” filed on Apr. 5, 2022. The disclosure of the foregoing application is incorporated herein by reference in its entirety for all purposes.

### BACKGROUND

Life sciences companies face unique challenges and opportunities when engaging with healthcare professionals (HCPs) and healthcare organizations (HCOs) globally. Different countries, states, and cities may have different healthcare regulations, and these regulations may change with time. Complying with global and local regulations for launching pharmaceutical products and maintaining the product on the market is evolving into a more time-consuming and challenging task.

### SUMMARY

Systems, methods, devices, and non-transitory, computer-readable storage media are disclosed for automatically recommending a compliance decision in response to a user's question or a search query associated with healthcare compliance. In some implementations, the decision can be recommended based on former decisions and/or insights provided by other users. In some implementations, a plurality of decisions are recommended to the user, and the user can select a decision among the plurality of decisions.

Systems, methods, devices, and non-transitory, computer-readable storage media are also disclosed for harvesting new or updated healthcare compliance requirements from a health authority website. In some implementations, robots harvest healthcare compliance regulation documents from the health authority website. A compliance regulation document classifier classifies compliance regulation documents into the first categories. The compliance requirement classifier identifies healthcare compliance requirements from the harvested compliance regulation documents and classifies healthcare compliance requirements into second categories. If the healthcare compliance requirements are new or updated, then the new compliance requirements or updated compliance requirements are stored in a database. In some implementations, a subject-matter expert (SME) can validate the first categories of healthcare compliance regulation documents and second categories of healthcare compliance requirements, and adjust the first categories or second categories if the validation fails (e.g., if the SME disagrees with first categories classified by the compliance regulation document classifier or second categories classified by the compliance requirement classifier). In some implementations, healthcare compliance concepts and/or terms can be extracted from healthcare compliance regulation documents and added to a healthcare compliance vocabulary database.

According to one innovative aspect of the present disclosure, in an aspect, a computer-implemented method includes receiving, by a machine learning model, a question associated with healthcare compliance from a user; identifying, by the machine learning model, a healthcare compliance regulation document associated with the question and one or more healthcare compliance requirements corresponding to the healthcare compliance regulation document; and recommending, by the machine learning model, a decision satisfying the one or more healthcare compliance requirements to the user.

Other aspects include apparatuses, systems, and computer programs for performing the actions of the aforementioned method.

The innovative method can include other optional features. For example, in some implementations, the method can further include identifying at least one precedent including a former decision made by another user associated with the question; and recommending the decision satisfying the one or more healthcare compliance requirements based on the at least one precedent.

In some implementations, wherein recommending the decision can include identifying at least one precedent including a former decision made by a former user associated with the question; identifying at least one insight provided by another former user; recommending a plurality of decisions to the user based on the at least one precedent and the at least one insight; and receiving a selection among the plurality of decisions from the user.

In some implementations, the method can include: receiving the healthcare compliance regulation document harvested from a health authority website; classifying the healthcare compliance regulation document into a first category; identifying the one or more healthcare compliance requirements from the healthcare compliance regulation document; classifying each healthcare compliance requirement into a second category; determining that each healthcare compliance requirement is a new healthcare compliance requirement or an updated healthcare compliance requirement; and in response to determination, storing the new healthcare compliance requirement or the updated healthcare compliance requirement in a database.

In some implementations, wherein the healthcare compliance regulation document is harvested from the health authority website by a robot.

In some implementations, the method can further include receiving, from a subject-matter expert (SME), a first validation for classification of the healthcare compliance regulation document into the first category; in response to the first validation, classifying the healthcare compliance regulation document into a third category; receiving, from the SME, a second validation for each healthcare compliance requirement into the second category; and in response to the second validation, classifying each healthcare compliance requirement into a fourth category.

In some implementations, the method can further include extracting at least one healthcare compliance term from the healthcare compliance regulation document; providing the at least one healthcare compliance term to a subject-matter expert (SME); and adding the at least one healthcare compliance term to a healthcare compliance vocabulary engine upon approval of the SME.

In some implementations, the method can further include training the machine learning model using the decision.

According to another innovative aspect of the present disclosure, in one aspect, the method can include receiving a healthcare compliance regulation document harvested by a robot from a health authority website; classifying the healthcare compliance regulation document into a first category; identifying one or more healthcare compliance requirements from the healthcare compliance regulation document; classifying each healthcare compliance requirement into a second category; determining that each healthcare compliance requirement is a new healthcare compliance requirement or an updated healthcare compliance requirement; and in response to determination, storing the new healthcare compliance requirement or the updated healthcare compliance requirement in a database.

Other aspects include apparatuses, systems, and computer programs for performing the actions of the aforementioned method.

The innovative method can include other optional features. For example, in some implementations, the method can further include receiving, from a subject-matter expert (SME), a first validation for classification of the healthcare compliance regulation document into the first category; in response to the first validation, classifying the healthcare compliance regulation document into a third category; receiving, from the SME, a second validation for each healthcare compliance requirement into the second category; and in response to the second validation, classifying each healthcare compliance requirement into a fourth category.

In some implementations, the method can further include extracting at least one healthcare compliance term from the healthcare compliance regulation document; providing the at least one healthcare compliance term to a subject-matter expert (SME); and adding the at least one healthcare compliance term to a healthcare compliance vocabulary engine upon approval of the SME.

In some implementations, the method can further include receiving a question associated with healthcare compliance from a user; identifying a healthcare compliance regulation associated with the question and the one or more healthcare compliance requirements corresponding to the healthcare compliance regulation; and recommending a decision satisfying the one or more healthcare compliance requirements to the user.

In some implementations, the method can further include identifying at least one precedent including a former decision made by another user associated with the question; and recommending the decision satisfying the one or more healthcare compliance requirements based on the at least one precedent.

In some implementations, wherein recommending the decision can include identifying at least one precedent including a former decision made by a former user associated with the question; identifying at least one insight provided by another former user; recommending a plurality of decisions to the user based on the at least one precedent and the at least one insight; and receiving a selection among the plurality of decisions from the user.

In an aspect, a computing system includes one or more processors coupled to a memory, the one or more processors configured to perform a method according to any of the aspects or embodiments described above.

In an aspect, a non-transitory computer readable medium stores instructions for causing a computing system to perform a method according to any of the aspects or embodiments described above.

The techniques described herein can provide various benefits. For instance, in some implementations, the user can automatically get a recommendation of a decision to satisfy healthcare compliance requirements, in response to the user's question or search query. The techniques can significantly improve the efficiency of making compliance decisions in different healthcare markets (different countries, states, and cities). The techniques can automatically obtain new or updated healthcare compliance requirements, so that the compliance decision can be made based on the newest healthcare compliance requirements.

The techniques can manage and transform regulatory data to minimize regulatory risk. The techniques are implemented on an open, scalable, and extendable architecture enabling easy integration of other systems, e.g., a labeling and structured content authoring regulatory system. The techniques can enable regulatory professionals to rapidly find regulatory information and generate valuable insights. The techniques can significantly increase quality and consistency of regulatory data while decreasing the associated workload considerably.

The details of one or more implementations are set forth in the accompanying drawings and the description below. Other features and advantages will be apparent from the description and drawings, and from the claims.

### BRIEF DESCRIPTION OF DRAWINGS

FIG. 1 is an example compliance intelligence system, according to some implementations of this disclosure.

FIG. 2 is a block diagram of an example compliance requirement classifier, according to some implementations of this disclosure.

FIG. 3 is a flow chart of an example method of recommending a compliance decision, according to some implementations of this disclosure.

FIG. 4 is a flow chart of an example method of selecting a compliance decision, according to some implementations of this disclosure.

FIG. 5 is a flow chart of an example method of automatically obtaining compliance requirements, according to some implementations of this disclosure.

FIG. 6 is a block diagram of an example computing system that can be used in connection with systems and methods described in this disclosure.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

Systems, methods, devices, and non-transitory, computer-readable storage media are disclosed for automatically recommending a compliance decision in response to a user's question or a search query associated with healthcare compliance. Systems, methods, devices, and non-transitory, computer-readable storage media are also disclosed for harvesting new or updated healthcare compliance requirements from a health authority website. In some implementations, robots harvest healthcare compliance regulation documents from the health authority website and identify healthcare compliance requirements from the harvested compliance regulation documents.

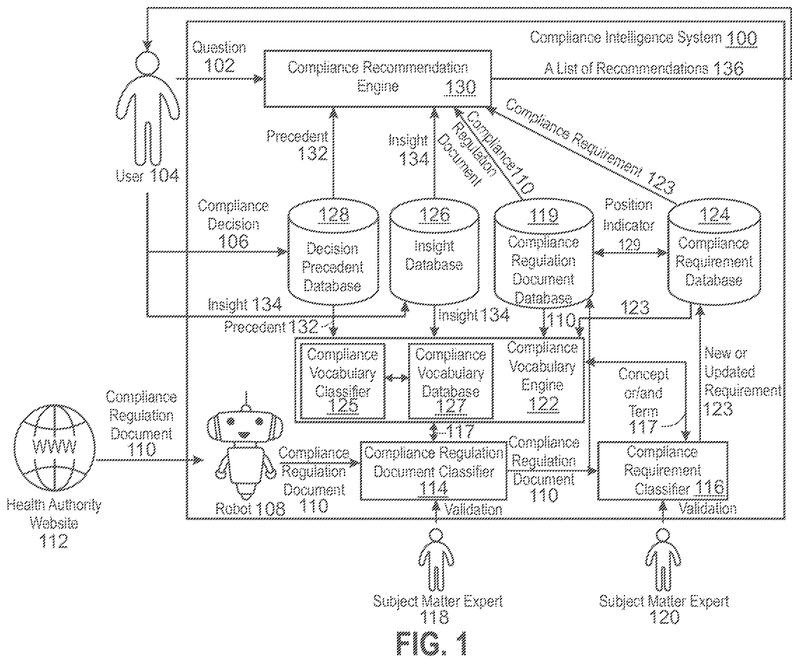

FIG. 1 shows an example compliance intelligence system 100 (also referred to as “Regulatory Digital Worker”). The example compliance intelligence system 100 is an integrated system for detection, intake, enrichment, management, and consumption of regulatory data (regulatory documents, regulatory requirements, and precedents). The example compliance intelligence system 100 can harvest compliance regulations and compliance requirements from the health authority website (e.g., periodically or upon request), and thus can get the latest compliance regulations and compliance requirements in a timely manner. The example compliance intelligence system 100 can also receive a question or a query 102 (e.g., What are the requirements for extending the shelf life of a particular pharmaceutical product in the European Union? What is the climatic zone for stability testing? How many lots are required to be tested for stability?) from a user 104 (e.g., a compliance professional of a pharmaceutical and medical technology (MedTech) company) and recommend a compliance decision 106 (e.g., providing stability data for two batches of the pharmaceutical product to apply for an extension of shelf life; Climatic Zone I in US, Climatic Zone II in European Union; Two lots required in US, three lots required in European Union) in response to the question 102. For example, the question or search query 102 and the recommended compliance decision 106 in reply can be shown in an artificial intelligence (AI) chatbot integrated with the example compliance intelligence system 100. The example compliance intelligence system 100 can provide a full audit trail and traceability from answers (recommended compliance decision 106) to source documents (compliance regulation documents 110) or questions 102.

The compliance intelligence system 100 includes a robot 108, configured to harvest compliance regulation documents 110 from one or more health authority web sites 112. The compliance regulation documents 110 can be global level (different countries), regional level (a particular region), or country-level (a particular country). In some implementations, a compliance regulation document includes one or more compliance requirements, one or more test conditions, a compliance process, a penalty in case of violation, and other regulatory information. The compliance regulation documents 110 are provided to compliance regulation document classifier 114, which classifies compliance regulation documents 110 into respective categories (first categories). For example, compliance regulation document classifier 114 can classify compliance regulation documents 110 based on a title, an abstract, keywords, etc., with reference to concepts/terms stored in the compliance vocabulary database 127. A subject matter expert (SME) 118 can validate the classification made by the compliance regulation document classifier 114. The SME 118 can adjust one or more categories if the validation fails. For example, the SME 118 reads compliance regulation documents 110 and determines whether the classification made by the compliance regulation document classifier 114 is accurate. If the classification is inaccurate, the SME 118 can adjust the one or more categories. The compliance regulation document classifier 114 is a type of machine learning algorithm used to assign a class label (e.g., categories) to a data input (e.g., compliance regulation documents). For example, the categories can be General Health Laws and Regulations, Nonclinical research, Clinical research, Health IT/Data Protection, Submissions, Packaging and Labelling, Pharmacovigilance and Risk Management, Quality Assurance, Chemistry Manufacturing & Controls (CMC), Advertising, Import, and Export, etc. In some implementations, the compliance regulation document classifier 114 is trained using supervised machine learning. Class labels (e.g., categories) adjusted by the SME 118 can be used to train the compliance regulation document classifier 114. For example, the compliance regulation document classifier 114 can be trained with a large number of documents, e.g., 10 million documents having more than 1400 document types.

The classified compliance regulation documents 110 are provided to compliance requirement classifier 116 and the compliance regulation document database 119. In some implementations, the compliance regulation document database 119 is a digital twin, which is a digital representation of compliance regulation documents 110. The classified compliance regulation documents 110 stored in the compliance regulation document database 119 include compliance regulation documents, metadata of each compliance regulation document, and a classification of each compliance regulation document. The metadata includes a source of a compliance regulation document (e.g., from the Food and Drug Administration (FDA), etc.), a date created, a date modified, a file size, a version, a market, a stage, a phase, a regulatory activity, a topic, a subtopic, a competent authority, a content type, etc. In some implementations, the compliance regulation documents 110 are fed into the compliance regulation document database 119 through containers.

The digital twin is a fully digital version of each piece of content (e.g., in a format such as: *.html, *.doc, *.pdf, etc.) with relevant metadata. The digital twin includes data that describes the content of each classified compliance regulation document, e.g., document construction/layout, section segments, embedded images, etc.). Any change to the content will automatically update the digital twin, which enables version control with full information lineage and inspection/auditability. The digital twin can keep the links across versions and allow the user to easily view the changes. Multiple versions of a compliance regulation document can be compared, with differences automatically highlighted.

The compliance requirement classifier 116 can extract requirements from the compliance regulation documents 110 and classify the extracted requirements into respective categories (second categories). For example, the compliance requirement classifier 116 can classify requirements based on their content (e.g., shelf life, the number of batches, the number of lots, etc.), with reference to concepts/terms stored in the compliance vocabulary database 127. A subject matter expert (SME) 120 can validate the classification made by the compliance requirement classifier 116. The SME 120 can adjust one or more categories if the validation fails. For example, the SME 120 reads requirements extracted from the compliance regulation documents 110 and determines whether the classification made by the compliance requirement classifier 116 is accurate. If the classification is inaccurate, the SME 120 can adjust the one or more categories. In some implementations, the SME 120 and the SME 118 can be the same SME or different SMEs. The compliance requirement classifier 116 is a type of machine learning algorithm used to assign a class label (e.g., categories) to a data input (e.g., regulations and/or requirements). In some implementations, the compliance requirement classifier 116 is trained using supervised machine learning. Class labels (e.g., categories) adjusted by the SME 120 can be used to train the compliance requirement classifier 116.

In some implementations, the example compliance intelligence system 100 further includes a compliance vocabulary engine 122 operating as a classification frame and a concepts/terms taxonomy database. The decision precedent database 128, insight database 126, compliance regulation document database 119, and compliance requirement database 124 can all communicate with the compliance vocabulary engine 122 for classification according to concepts/terms taxonomy (concept are higher than terms in a hierarchy). Taxonomy refers to a hierarchical structure of categories or labels that are used to classify data. The compliance vocabulary engine 122 includes compliance vocabulary database 127 and compliance vocabulary classifier 125. The compliance vocabulary database 127 can store concepts and/or terms extracted from compliance regulation documents and compliance requirements. The compliance vocabulary classifier 125 can classify the input data (e.g., precedent 132, insight 134, compliance regulation document 110, or compliance requirement 123) according to concepts and/or terms taxonomy. For example, the compliance vocabulary classifier 125 can determine a hierarchical level of the keywords of the input data in the concepts and/or terms taxonomy. The concepts and/or terms stored in the compliance vocabulary database 127 are reference data for classifying the input data and connecting the decision precedent database 128, insight database 126, compliance regulation document database 119, and compliance requirement database 124 with each other.

For example, when a user 104 provides an insight 134, the concept and terms of the insight 134 are used to map the insight 134 to one or more precedents 132 in the decision precedent database 128, one or more compliance regulations in the compliance regulation document database 119, and one or more compliance requirements in the compliance requirement database 124. The concepts and terms of the insight 134 are also stored in the compliance vocabulary database 127 if there are no such concepts and terms in the compliance vocabulary database 127.

In some implementations, the compliance requirement classifier 116 can extract compliance concepts and/or terms 117 from compliance regulation documents 110. If they are new concepts and/or terms, they are stored in the compliance vocabulary database 127. In some implementations, the compliance concepts and/or terms 117 (e.g., regulatory activity type, regulatory submission type, regulatory contribution type, regulatory content type, etc.) are stored in the compliance vocabulary database 127 upon approval of an SME. The SME can be SME 120 or a different SME. The compliance regulation documents 110 in different jurisdictions (e.g., different countries, states, counties, cities, towns, etc.) may have different concepts and/or terms 117.

In some implementations, the compliance requirement classifier 116 can split each compliance regulation document 110 into a plurality of fragments (e.g., paragraphs, sentences), and each fragment is subject to natural language processing (NLP). The compliance requirement classifier 116 can identify the content of each fragment (each paragraph, each sentence) for classification of the requirements extracted in the compliance regulation document 110. In some implementations, the plurality of fragments are linked to maintain a content structure of each compliance regulation document 110. The linked plurality of fragments and metadata can create a digital twin of each compliance regulation document 110.

In some implementations, NLP is based on a Linguamatics platform and a global, best-in-class system for deploying innovative NLP-based text mining for high-value knowledge discovery and decision support. NLP uses a hybrid model, e.g., NLP leverages statistical models, linguistic models, and machine learning with a rules-based query development interface. The NLP can normalize terminology across regulatory content utilizing queries and ontologies and support multiple interfaces to suit a variety of users for data understanding and exploration. NLP enables the development/expansion of ontologies and NLP queries to enrich, extract and normalize key data attributes and fragments. NLP allows the incorporation of linguistic contexts such as relationships, negations, and phrases. The NLP also accommodates spelling and OCR corrections, glyphs, dialects, and more. NLP provides a fully featured user interface/data science workbench for data scientists to enable ad hoc querying and analysis, as well as multi-query capabilities for more complex analysis or extraction. Data scientists can leverage a broad range of resource queries and ontologies. NLP comes with extensive RESTful application programming interfaces (APIs) and software development kits (SDKs) for Python, JavaScript, and Java, enabling potential automation and integration beyond the scope of a request for information (RFI).

In some implementations, the compliance requirement classifier 116 can determine whether each of the compliance requirements extracted from a compliance regulation document 110 is a new compliance requirement, or an updated or changed compliance requirement 123. The new compliance requirements or updated compliance requirements 123 can be stored in the compliance requirement database 124. For example, the compliance requirement classifier 116 can check the metadata of each compliance requirement and/or content of each compliance requirement, and determine whether each compliance requirement is a new compliance requirement or an updated compliance requirement. In some implementations, a sentence of a compliance regulation document 110, a paragraph of the compliance regulation document 110, a compliance requirement of the compliance regulation document 110, or the compliance regulation document 110 can have its own metadata. For example, the metadata can be extracted using Named Entity Recognition and NLP.

In some implementations, a compliance regulation document 110 stored in the compliance regulation document database 119 can be associated with corresponding compliance requirements by a position indicator or a position pointer 129. The position indicator 129 can indicate a position of a compliance requirement within a compliance regulation document, e.g., one or more of page number, section number, paragraph number, column number, and line number, indicating the position of the compliance requirement within a compliance regulation document. In some implementations, the position indicator 129 includes a position of a compliance regulation document within the compliance regulation document database 119, a position of a compliance requirement within the compliance requirement database 124, and a position of the compliance requirement within the compliance regulation document.

In some implementations, the example compliance intelligence system 100 further includes an insight database 126, which stores historical insights provided by former users. The insights are personal opinions, recommendations, etc., about the regulations and/or requirements.

In some implementations, the example compliance intelligence system 100 further includes a decision precedent database 128, which stores historical compliance decisions made or selected by former users.

In some implementations, the example compliance intelligence system 100 further includes a compliance recommendation engine 130. The compliance recommendation engine 130 can provide a list of compliance decision recommendations 136 to the user 104 in response to a compliance question or query 102 of the user 104. The list of compliance decision recommendations 136 can be provided based on precedent 132 from the decision precedent database 128, compliance requirements 123 from the compliance requirement database 124, and/or insights 134 from the insight database 126. The list of compliance decision recommendations 136 can include a combination of any of precedent 132, compliance requirements 123, and/or insights 134. The user 104 selects a compliance decision 106 from the list of compliance decision recommendations 136. The selected compliance decision 106 can be stored in the decision precedent database 128 as a precedent 132.

In some implementations, the compliance recommendation engine 130 is a machine learning model, which was trained using a large number of prior compliance decisions. The compliance recommendation engine 130 can also be trained using the precedent 132 from the decision precedent database 128, the compliance requirements 123 from the compliance requirement database 124, and/or insights 134 from the insight database 126. During the training stage, an SME (e.g., SME 118, SME 120, or a different SME) confirms or adjusts a proposed decision 106 output by the compliance recommendation engine 130. The compliance recommendation engine 130 continuously learns from interactions with the SME. The compliance recommendation engine 130 can be a supervised machine learning model (e.g., convolutional neural network (CNN). For example, the compliance recommendation engine 130 can run a machine learning algorithm, such as Logistic Regression, Support Vector Machines (SVM), Naive Bayes, Decision Trees, Linear Regression, k Nearest Neighbors (kNN) technique, Random Forest, or Boosting algorithms (such as Gradient Boosting Machine, XGBoost, or LightGBM, etc.), etc.

In some implementations, while the compliance intelligence system 100 has the ability to connect with third-party tools, such as Tableau, Spotfire, and Microstrategy, etc., the possibility of managing all aspects within one platform has key benefits to ongoing security, auditing, governance, and the ability to leverage existing cluster resources to their fullest capabilities. Data of the compliance intelligence system 100 is accessible through a variety of standard protocols and tooling, such as R, Python libraries, Open Database Connectivity (ODBC)/Java Database Connectivity (JDBC), or native Hadoop libraries, Hadoop Distributed File System (HDFS) connectivity, etc.

In some implementations, the harvested compliance regulation documents 110 are digitalized. For example, each compliance regulation document 110 is assessed to determine a quality grade, a document type, and the language of each compliance regulation document 110 and identify embedded images of each compliance regulation document 110. Each compliance regulation document 110 is subjected to scan, segmentation (fragmentation), Optical Character Recognition (OCR), and ontology-based correction. Each compliance regulation document 110 is structured into Extensible Markup Language (XML) components, matching a specific document type using Text, Construct, and Image standards. The content digitalization is described in a US patent application No. U.S. Ser. No. 16/289,729, filed on Mar. 1, 2019, entitled “Automated Classification and Interpretation of Life Science Documents,” which is incorporated herein by reference in its entirety for all purposes.

In some implementations, the compliance intelligence system 100 further includes a translation system that delivers multilingual content quickly, accurately, and securely, using advanced artificial intelligence and neural machine technology. The compliance intelligence system 100 can provide high-quality translation for regulatory data (e.g., regulation terminologies, regulation requirements, paragraphs in a regulation document, etc.). The translation system is described in a US patent application No. U.S. Ser. No. 17/728,561, filed on Apr. 25, 2022, entitled “Automation-Enhanced Translation Workflow,” which is incorporated herein by reference in its entirety for all purposes.

In some implementations, the compliance intelligence system 100 further includes a structured content authoring system (SCA). For example, Smart label, as an example use case for SCA, is an enterprise platform for label request, label and artwork, label planning and management, (collaborative) authoring, translation, review and approval, submission, and artwork tracking. Smart label empowers labeling professionals with a tool that provides visibility and control over a label request plan and label journeys with intuitive GANTT planning and tracking tools, including one-click analytics to instantly view insights such as actual dates versus planned dates and real time variance. Users can create, assess, and approve label plans and author and execute global labels, which can then be sent to affiliates for localization and local artwork creation.

In some implementations, using pre-defined rules, the SCA will identify suitable, reusable fragments, classify them and embed specific semantic tags, and re-use rules to assist in the automation of draft label material and semantic authoring of less templated material. In some implementations, an assembly is generated from approved ‘assembly’ templates, which can also define a style and a format of the text. Authors can select fragments and elements that are recommended to them, based on the piece they are authoring.

In some implementations, co-authoring is also provided. Fragments or full sections of an assembly document can be routed to an SME for completion. Once authoring is complete, the author can send the assembly document for peer review and/or submit it for draft review and approval. In some implementations, the fragments and assemblies are adaptive, and can be published in traditional formats, such as Word or PDF.

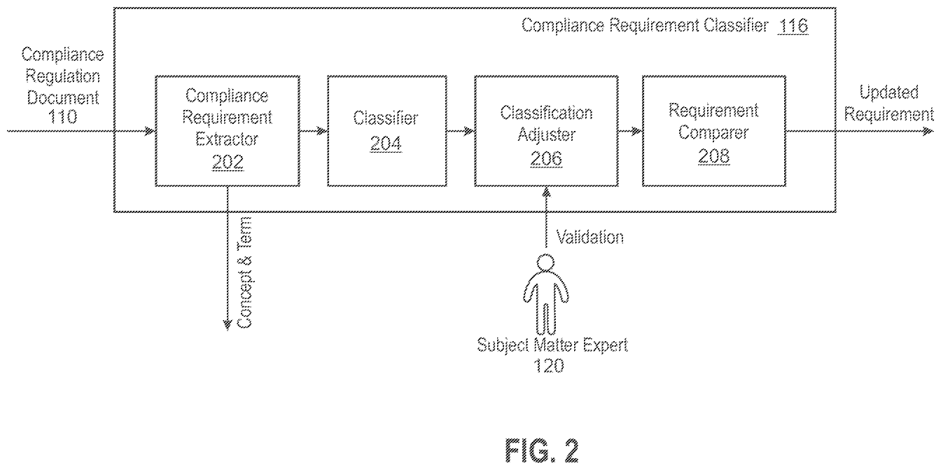

FIG. 2 is a block diagram of an example compliance requirement classifier 116. In some implementations, the compliance requirement classifier 116 includes a compliance requirement extractor 202, classifier 204, classification adjuster 206, and requirement comparer 208. The compliance requirement extractor 202 is configured to extract compliance requirements from compliance regulation documents 110 through natural language processing (NLP). For example, the compliance requirement extractor 202 can extract compliance requirements using a bag-of-words (BoW) technique. In some implementations, the compliance requirement extractor 202 can perform tokenization to split each compliance regulation document 110 into a plurality of concepts and/or terms, which can be stored in compliance vocabulary database 127 of FIG. 1. The classifier 204 is configured to classify extracted compliance requirements into respective categories. The classification adjuster 206 is configured to adjust categories determined by the classifier 204 if validation by SME 120 fails (SME 120 disagrees with categories determined by the classifier 204). In some implementations, the requirement comparer 208 is configured to compare a compliance requirement with compliance requirements stored in the compliance requirement database 124. For example, the requirement comparer 208 can perform a textual and semantic similarity comparison, a word-to-word comparison, etc. The requirement comparer 208 can obtain a degree of similarity between the compliance requirement and the compliance requirements stored in the compliance requirement database 124, and determine whether the compliance requirement is new, updated, or the same with respect to compliance requirements stored in the compliance requirement database 124. If the compliance requirement is new (not found in the compliance requirement database 124) or updated (different from the corresponding requirement in the compliance requirement database 124), the new or updated compliance requirement is stored in the compliance requirement database 124. For example, if a particular percentage (e.g., 70%) of words or terms of a compliance requirement is different from any compliance requirement in the compliance requirement database 124, this compliance requirement is considered a new compliance requirement. For example, if a particular percentage (e.g., 50%) of words or terms of a compliance requirement is the same as any compliance requirement in the compliance requirement database 124, this compliance requirement is considered an updated compliance requirement. For example, if the compliance requirement is the same as a particular compliance requirement in the compliance requirement database 124, this compliance requirement is ignored or deleted.

In some implementations, the requirement comparer 208 can compare the metadata of a compliance requirement with metadata of compliance requirements stored in the compliance requirement database 124 to determine if the compliance requirement is new, updated, or the same as any compliance requirement stored in the compliance requirement database 124. For example, the requirement comparer 208 can compare version information included in the metadata.

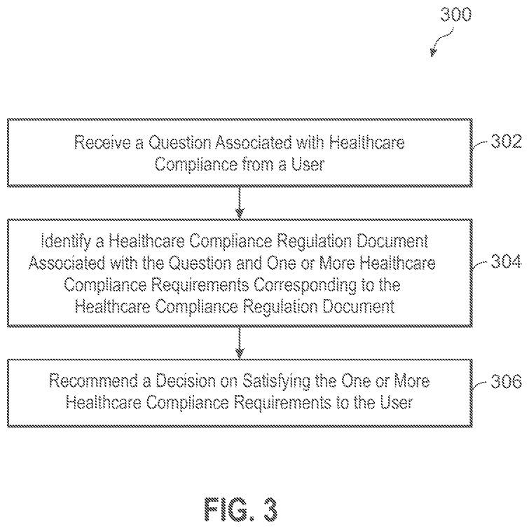

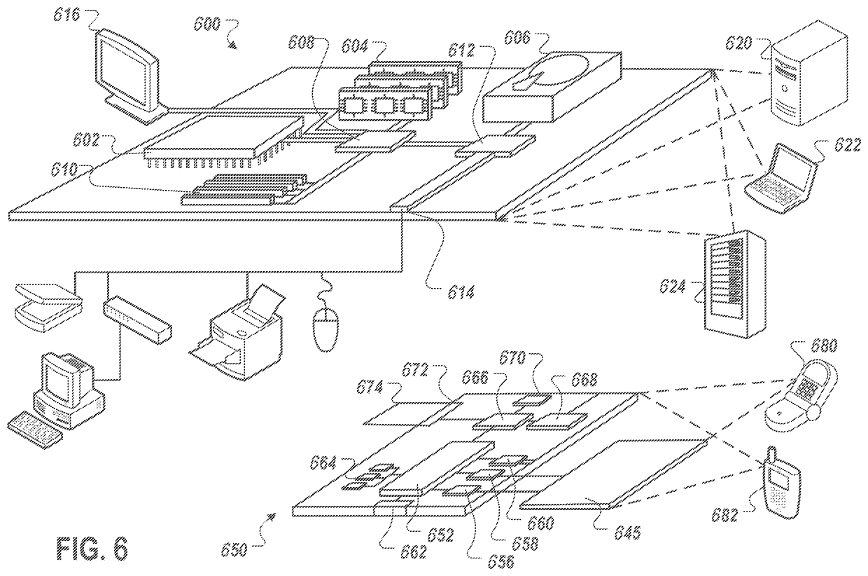

FIG. 3 is a flow chart of an example method 300 of recommending a compliance decision. The example method 300 can be implemented by a data processing apparatus, a computer-implemented system, or a computing system, such as a computing device 600, 650 as shown in FIG. 6 or the example compliance intelligence system 100 as shown in FIG. 1. In some implementations, a computing system can be a system of one or more computers, located in one or more locations, and programmed appropriately in accordance with this disclosure. For example, a computing device 600, 650 in FIG. 6, appropriately programmed, can perform the example method 300. In some implementations, the example method 300 can be implemented on or in conjunction with a Digital Signal Processor (DSP), Field Programmable Gate Array (FPGA), processor, controller, and/or a hardware semiconductor chip, etc.

At block 302, a computing device (e.g., the computing device 600, 650 of FIG. 6) receives a question or query (e.g., question 102 of FIG. 1) associated with healthcare compliance from a user (e.g., user 104 of FIG. 1). For example, the user asks a question “what are the requirements of extending a shelf life of Ibuprofen in Australia?” For another example, the user inputs a query “Argentina stability data for Methadone.”

At block 304, the computing device identifies a healthcare compliance regulation document associated with the question or query, and one or more healthcare compliance requirements corresponding to the healthcare compliance regulation document. In some implementations, the computing device identifies keywords from the question or query, e.g., “shelf life,” “Ibuprofen,” “Australia” for the question or “Argentina,” “stability data,” “Methadone” for the query using natural language processing. The computing device can identify concepts of these keywords. For example, “shelf life” and “stability data” correspond to the concept “regulatory activity,” “Ibuprofen” and “Methadone” correspond to the concept “pharmaceutical product,” “Australia” and “Argentina” correspond to the concept “geography.” The computing device can identify a compliance regulation document stored in a first database (the compliance regulation document database 119 of FIG. 1) and its corresponding compliance requirements from a second database (e.g., the compliance requirement database 124 of FIG. 1) through, e.g., concept matching, and a degree of textual and semantic similarity based on relevance. For example, the computing device identifies a compliance regulation document related to shelf life of Ibuprofen in Australia or stability data for Methadone in Argentina, and this compliance regulation document includes compliance requirements, e.g., “one year” for the question, or “three batches of stability data” and “non-pilot stability data” for the query. In some implementations, the computing device can identify a compliance requirement if concepts of the keywords of the question or query and concepts of keywords of the compliance requirement are the same. The compliance vocabulary classifier 125 can determine a first hierarchical level of the keywords of the question or query in a concepts/terms taxonomy, and identify a compliance regulation requirement having a second hierarchical level closest to the first hierarchical level in the concepts/terms taxonomy. In some implementations, a plurality of relevant compliance regulation documents and corresponding compliance requirements are identified by the computing device. The plurality of relevant compliance regulation documents and corresponding compliance requirements can be presented to the user in a list (or any other forms), which is ranked according to relevance with respect to the question or query (e.g., question 102 of FIG. 1) received at block 302.

At block 306, the computing device recommends a compliance decision (an answer to the question or query) satisfying the one or more healthcare compliance requirements to the user. The compliance decision is provided as an output from the computing device. A trained machine learning model (e.g., compliance recommendation engine 130 of FIG. 1) can recommend a compliance decision in response to the question or query. The machine learning model can be trained by a large number of prior decisions (e.g., from the decision precedent database 128), concepts and terms (e.g., from compliance vocabulary database 127), compliance regulation documents (e.g., from the compliance regulation document database 119), and compliance requirements (e.g., from the compliance requirement database 124) and insights (e.g., from the insight database 126). The training process involves initializing some random values for each of the training matrixes and attempting to predict the output of the input data using the initial random values. In the beginning, the error will be large, but by comparing the model's prediction with the correct output (e.g., labeled by SME 118, 120), the machine learning model is able to adjust the weights and biases values until having a good predicting model.

For example, the computing device recommends a compliance decision “one year shelf life of Ibuprofen in Australia.” For another example, the computing device recommends a compliance decision “providing three batches of non-pilot stability data for approval.” This compliance decision describes how to satisfy the healthcare compliance requirements. The decision enables the user to take action, e.g., designing a clinical trial, etc. For example, the user can prepare three batches of non-pilot stability data in accordance with the decision.

In some implementations, the recommended decision can be stored in the decision precedent database 128 as precedent for continuously training the compliance recommendation engine 130. In some implementations, the user (e.g., the user 104 of FIG. 1) can provide an insight (e.g., opinions, suggestions, know-how, expertise, etc.) about the recommended decision, and the insight can be stored in the insight database 126 for continuously training the compliance recommendation engine 130. For example, an insight “more batches and non-pilot data can expedite approval” can be provided by the user 104 and stored in the insight database 126.

In some implementations, the decision precedent database 128, insight database 126, compliance regulation document database 119, compliance requirement database 124, and/or compliance vocabulary database 127 may include confidential data. For example, Cloudera distribution of Hadoop can be used to store confidential data.

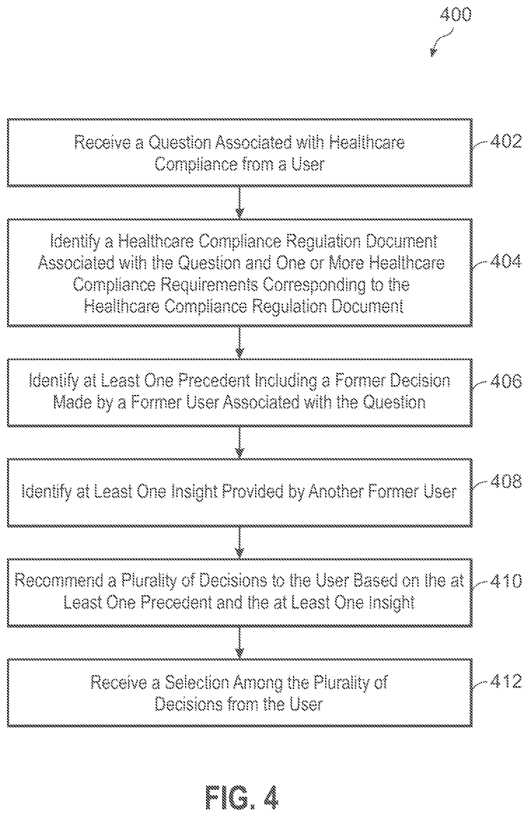

FIG. 4 is a flow chart of an example method 400 of selecting a compliance decision. The example method 400 can be implemented by a data processing apparatus, a computer-implemented system, or a computing system, such as a computing device 600, 650 as shown in FIG. 6 or the example compliance intelligence system 100 as shown in FIG. 1. In some implementations, a computing system can be a system of one or more computers, located in one or more locations, and programmed appropriately in accordance with this disclosure. For example, a computing device 600, 650 in FIG. 6, appropriately programmed, can perform the example method 400. In some implementations, the example method 400 can be implemented on or in conjunction with a Digital Signal Processor (DSP), Field Programmable Gate Array (FPGA), processor, controller, and/or a hardware semiconductor chip, etc.

At block 402, a computing device (e.g., the computing device 600, 650 of FIG. 6) receives a question (e.g., question 102 of FIG. 1) associated with healthcare compliance from a user (e.g., user 104 of FIG. 1). For example, the user asks a question “what are the requirements of extending a shelf life of Tylenol in Saudi Arabia?”

At block 404, the computing device identifies a healthcare compliance regulation document (e.g., from compliance regulation document database 119) associated with the question and one or more healthcare compliance requirements (e.g., from compliance requirement database 124) corresponding to the healthcare compliance regulation document. For example, the computing device identifies a compliance regulation document related to the shelf life of Tylenol in Saudi Arabia, and this compliance regulation document includes compliance requirements “two batches of stability data” and “pilot stability data is acceptable.”

At block 406, the computing device identifies at least one precedent including a former decision made by a former user associated with the question. In some implementations, the computing device can identify one or more precedents from a decision precedent database (e.g., decision precedent database 128 of FIG. 1). The one or more precedents are identified based on their relevance to the question asked by the user at block 402, e.g., through concept matching or a degree of textual and semantic similarity based on relevance. For example, the computing device retrieves a former decision “providing four batches of non-pilot stability data for approval.” This former decision can expedite the approval of extending the shelf life in Saudi Arabia.

At block 408, the computing device identifies at least one insight provided by a former user. The computing device can identify one or more insights from the insight database (e.g., insight database 126 of FIG. 1). The one or more insights are identified based on their relevance to the question asked by the user at block 402, e.g., through concept matching or a degree of textual and semantic similarity based on relevance. The relevance can be determined based on, e.g., the number of matched keywords. For example, an insight associated with the question is “more than two batches of stability data can expedite the approval.” The former user at block 408 can be the same as or different from the former user at block 406.

At block 410, the computing device recommends a plurality of decisions to the user, based on the at least one precedent and the at least one insight. For example, the computing device recommends “providing two batches of non-pilot stability data for approval,” “providing two batches of pilot stability data for approval,” “providing three batches of non-pilot stability data for approval,” “providing three batches of pilot stability data for approval,” “providing four batches of non-pilot stability data for approval,” “providing four batches of pilot stability data for approval.” The plurality of decisions provide information on how to satisfy the healthcare compliance requirements.

In some implementations, each of the decisions is assigned a confidence score indicating the similarity degree between each decision and the compliance requirements to be satisfied. The similarity degree can be determined based on, e.g., the number of matched keywords, matched concepts and terms, and textual and semantic similarity. The plurality of decisions can be presented to the user in a list (or any other forms), which is ranked according to the confidence scores.

In some implementations, precedent(s) and insight(s) can also be presented to the user, together with the plurality of decisions. Each decision corresponds to its precedent(s) and insight(s).

At block 412, the computing device receives a selection among the plurality of decisions from the user. The user selects a compliance decision from the plurality of decisions. For example, the user selects a compliance decision “providing three batches of non-pilot stability data for approval” from the plurality of compliance decisions. Even though the decision “providing two batches of pilot stability data for approval” may meet the compliance requirements for stability data in Saudi Arabia, the user selects “providing three batches of non-pilot stability data for approval” to expedite approval based on the former decision at block 406 and the insight at block 408.

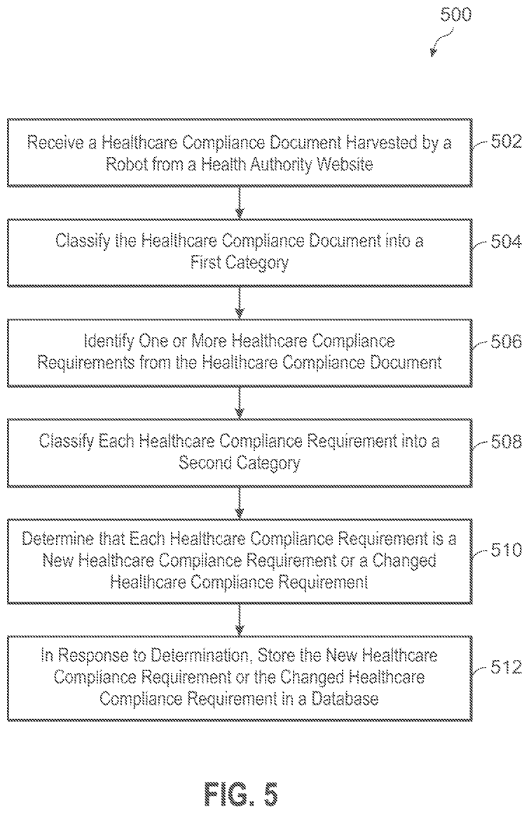

FIG. 5 is a flow chart of an example method 500 of automatically obtaining compliance requirements. The example method 500 can be implemented by a data processing apparatus, a computer-implemented system, or a computing system, such as a computing device 600, 650 as shown in FIG. 6 or the example compliance intelligence system 100 as shown in FIG. 1. In some implementations, a computing system can be a system of one or more computers, located in one or more locations, and programmed appropriately in accordance with this disclosure. For example, a computing device 600, 650 in FIG. 6, appropriately programmed, can perform the example method 500. In some implementations, the example method 500 can be implemented on or in conjunction with a Digital Signal Processor (DSP), Field Programmable Gate Array (FPGA), processor, controller, and/or a hardware semiconductor chip, etc.

At block 502, the computing device receives a healthcare compliance regulation document harvested by a robot (e.g., robot 108 of FIG. 1) from a health authority website. In some implementations, a large number of robots (e.g., about 500 robots) continuously monitor health authority websites of different countries, states, counties, cities, towns, etc., and periodically (e.g., every 12 hours) obtain compliance regulation documents and related metadata from the health authority websites (e.g., a website of US Food and Drug Administration). In some implementations, different robots may monitor websites in different jurisdictions, and each robot may have a different frequency (e.g., hourly, daily, weekly, etc.) of harvesting data.

At block 504, the computing device classifies the healthcare compliance regulation document into a first category. For example, the computing device classifies each healthcare compliance regulation document using a compliance regulation document classifier (e.g., compliance regulation document classifier 114 of FIG. 1). The first category can be, e.g., Fees, Device Classification, Quality Systems Requirements, Marketing Approval/Clearance Applications, Establishment Registration, Compassionate Use, Adverse Event Reporting, Recalls (Field Actions), Device Tracking, Inspection of Manufacturing Sites, Sale to Consumers, etc. The compliance regulation document classifier can be a machine learning model and use a machine learning algorithm, e.g., a Naive Bayes classifier, a decision tree, artificial neural networks (ANNs), a support vector machine (SVM), or K-nearest neighbor (KNN), logistic regression, random forest, etc.

At block 506, the computing device identifies one or more healthcare compliance requirements from the healthcare compliance regulation document. For example, the computing device extracts healthcare compliance requirements from each healthcare compliance regulation document using a compliance requirement extractor (e.g., compliance requirement extractor 202 of FIG. 2). In an example, the compliance requirement extractor can extract compliance requirements through concept matching or a degree of textual and semantic similarity based on relevance. The compliance requirement extractor can split each compliance regulation document into a plurality of concepts and/or terms for analysis using natural language processing.

At block 508, the computing device classifies each healthcare compliance requirement into a second category. For example, the computing device classifies each healthcare compliance requirement using a classifier (e.g., the classifier 204 of FIG. 2). The second category can be, e.g., Target Identification, Compound Screening, Drug design, Assay development, GLP, Renewals, Withdrawals, Marketing Status Change, Post-approval Safety, Expanded Access Use/Named Patient Use Safety, Clinical trials Safety, Storage and Distribution, etc. The classifier can be a machine learning model and use a machine learning algorithm, e.g., a Naive Bayes classifier, a decision tree, artificial neural networks (ANNs), a support vector machine (SVM), or K-nearest neighbor (KNN), logistic regression, random forest, etc.

At block 510, the computing device determines that each healthcare compliance requirement is a new healthcare compliance requirement or an updated healthcare compliance requirement. For example, the computing device can compare each compliance requirement (and/or metadata associated with each compliance requirement) with compliance requirements (and/or metadata associated with compliance requirements) stored in a database (the compliance requirement database 124 of FIG. 1), using a requirement comparer (e.g., requirement comparer 208 of FIG. 2).

At block 512, in response to the determination at block 510, the computing device stores the new healthcare compliance requirement or the updated healthcare compliance requirement in the database (compliance requirement database 124 of FIG. 1).

FIG. 6 is a block diagram of computing devices 600, 650 that may be used to implement the systems and methods described in this disclosure, either as a client or as a server, a cloud server, or multiple servers. Computing device 600 and 650 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations described and/or claimed in this document.

Computing device 600 includes a processor 602, memory 604, a storage device 606, a high-speed interface 608 connecting to memory 604 and high-speed expansion ports 610, and a low-speed interface 612 connecting to low-speed bus 614 and storage device 606. Each of the components 602, 604, 606, 608, 610, and 612, are interconnected using various busses, and may be mounted on a common motherboard or in other manners as appropriate. The processor 602 can process instructions for execution within the computing device 600, including instructions stored in the memory 604 or on the storage device 606 to display graphical information for a GUI on an external input/output device, such as display 616 coupled to high-speed interface 608. In other implementations, multiple processors and/or multiple buses may be used, as appropriate, along with multiple memories and types of memory. Also, multiple computing devices 600 may be connected, with each device providing portions of the necessary operations, e.g., as a server bank, a group of blade servers, or a multi-processor system.

The memory 604 stores information within the computing device 600. In one implementation, the memory 604 is a computer-readable medium. In one implementation, the memory 604 is a volatile memory unit or units. In another implementation, the memory 604 is a non-volatile memory unit or units.

The storage device 606 is capable of providing mass storage for the computing device 600. In one implementation, the storage device 606 is a computer-readable medium. In various different implementations, the storage device 606 may be a floppy disk device, a hard disk device, an optical disk device, a tape device, a flash memory or other similar solid-state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer- or machine-readable medium, such as the memory 604, the storage device 606, or memory on processor 602.

The high-speed controller 608 manages bandwidth-intensive operations for the computing device 600, while the low-speed controller 612 manages lower bandwidth-intensive operations. Such allocation of duties is exemplary only. In one implementation, the high-speed controller 608 is coupled to memory 604, display 616, e.g., through a graphics processor or accelerator, and to high-speed expansion ports 610, which may accept various expansion cards (not shown). In the implementation, low-speed controller 612 is coupled to the storage device 606 and low-speed expansion port 614. The low-speed expansion port, which may include various communication ports, e.g., USB, Bluetooth, Ethernet, wireless Ethernet, may be coupled to one or more input/output devices, such as a keyboard, a pointing device, a scanner, or a networking device such as a switch or router, e.g., through a network adapter.

The computing device 600 may be implemented in a number of different forms, as shown in the figure. For example, it may be implemented as a standard server 620, or multiple times in a group of such servers. It may also be implemented as part of a rack server system 624. In addition, it may be implemented in a personal computer such as a laptop computer 622. Alternatively, components from computing device 600 may be combined with other components in a mobile device (not shown), such as device 650. Each of such devices may contain one or more of computing device 600, 650, and an entire system may be made up of multiple computing devices 600, 650 communicating with each other.

Computing device 650 includes a processor 652, memory 664, an input/output device such as a display 654, a communication interface 666, and a transceiver 668, among other components. The device 650 may also be provided with a storage device, such as a Microdrive or other device, to provide additional storage. Each of the components 650, 652, 664, 654, 666, and 668, are interconnected using various buses, and several of the components may be mounted on a common motherboard or in other manners as appropriate.

The processor 652 can process instructions for execution within the computing device 650, including instructions stored in the memory 664. The processor may also include separate analog and digital processors. The processor may provide, for example, for coordination of the other components of the device 650, such as control of user interfaces, applications run by device 650, and wireless communication by device 650.

Processor 652 may communicate with a user through control interface 658 and display interface 656 coupled to a display 654. The display 654 may be, for example, a TFT LCD display or an OLED display, or other appropriate display technology. The display interface 656 may include appropriate circuitry for driving the display 654 to present graphical and other information to a user. The control interface 658 may receive commands from a user and convert them for submission to the processor 652. In addition, an external interface 662 may be provided in communication with processor 652, so as to enable near area communication of device 650 with other devices. External interface 662 may provide, for example, for wired communication, e.g., via a docking procedure, or for wireless communication, e.g., via Bluetooth or other such technologies.

The memory 664 stores information within the computing device 650. In one implementation, the memory 664 is a computer-readable medium. In one implementation, the memory 664 is a volatile memory unit or units. In another implementation, the memory 664 is a non-volatile memory unit or units. Expansion memory 674 may also be provided and connected to device 650 through expansion interface 672, which may include, for example, a SIMM card interface. Such expansion memory 674 may provide extra storage space for device 650, or may also store applications or other information for device 650. Specifically, expansion memory 674 may include instructions to carry out or supplement the processes described above, and may include secure information also. Thus, for example, expansion memory 674 may be provided as a security module for device 650, and may be programmed with instructions that permit secure use of device 650. In addition, secure applications may be provided via the SIMM cards, along with additional information, such as placing identifying information on the SIMM card in a non-hackable manner.

The memory may include for example, flash memory and/or MRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 664, expansion memory 674, or memory on processor 652.

Device 650 may communicate wirelessly through communication interface 666, which may include digital signal processing circuitry where necessary. Communication interface 666 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 668. In addition, short-range communication may occur, such as using a Bluetooth, Wi-Fi, or other such transceiver (not shown). In addition, GPS receiver module 670 may provide additional wireless data to device 650, which may be used as appropriate by applications running on device 650.

Device 650 may also communicate audibly using audio codec 660, which may receive spoken information from a user and convert it to usable digital information. Audio codec 660 may likewise generate audible sound for a user, such as through a speaker, e.g., in a handset of device 650. Such sound may include sound from voice telephone calls, may include recorded sound, e.g., voice messages, music files, etc., and may also include sound generated by applications operating on device 650.

The computing device 650 may be implemented in a number of different forms, as shown in the figure. For example, it may be implemented as a cellular telephone 680. It may also be implemented as part of a smartphone 682, personal digital assistant, or other similar mobile device.

Various implementations of the systems and techniques described here can be realized in digital electronic circuitry, integrated circuitry, specially designed ASICs, computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs, also known as programs, software, software applications or code, include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back-end component, e.g., as a data server, or that includes a middleware component such as an application server, or that includes a front-end component such as a client computer having a graphical user interface or a Web browser through which a user can interact with an implementation of the systems and techniques described here, or any combination of such back-end, middleware, or front-end components. The components of the system can be interconnected by any form or medium of digital data communication such as, a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

Memory stores program instructions and data used by the processor of the intrusion detection panel. The memory may be a suitable combination of random access memory and read-only memory, and may host suitable program instructions (e.g. firmware or operating software), and configuration and operating data and may be organized as a file system or otherwise. The program instructions stored in the memory of the panel may store software components allowing network communications and establishment of connections to the data network.

Program instructions stored in the memory, along with configuration data may control overall operation of the system. Server computer systems include one or more processing devices (e.g., microprocessors), a network interface and a memory (all not illustrated). Server computer systems may physically take the form of a rack mounted card and may be in communication with one or more operator terminals (not shown).

All or part of the processes described herein and their various modifications (hereinafter referred to as “the processes”) can be implemented, at least in part, via a computer program product, i.e., a computer program tangibly embodied in one or more tangible, physical hardware storage devices that are computer and/or machine-readable storage devices for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Actions associated with implementing the processes can be performed by one or more programmable processors executing one or more computer programs to perform the functions of the calibration process. All or part of the processes can be implemented as, special purpose logic circuitry, e.g., an FPGA (field programmable gate array) and/or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only storage area or a random access storage area or both. Elements of a computer (including a server) include one or more processors for executing instructions and one or more storage area devices for storing instructions and data. Generally, a computer will also include, or be operatively coupled to receive data from, or transfer data to, or both, one or more machine-readable storage media, such as mass storage devices for storing data, e.g., magnetic, magneto-optical disks, or optical disks.

Tangible, physical hardware storage devices that are suitable for embodying computer program instructions and data include all forms of non-volatile storage, including by way of example, semiconductor storage area devices, e.g., EPROM, EEPROM, and flash storage area devices; magnetic disks, e.g., internal hard disks or removable disks; magneto-optical disks; and CD-ROM and DVD-ROM disks and volatile computer memory, e.g., RAM such as static and dynamic RAM, as well as erasable memory, e.g., flash memory.

In addition, the logic flows depicted in the figures do not require the particular order shown, or sequential order, to achieve desirable results. In addition, other actions may be provided, or actions may be eliminated, from the described flows, and other components may be added to, or removed from, the described systems. Likewise, actions depicted in the figures may be performed by different entities or consolidated.

Elements of different embodiments described herein may be combined to form other embodiments not specifically set forth above. Elements may be left out of the processes, computer programs, Web pages, etc. described herein without adversely affecting their operation. Furthermore, various separate elements may be combined into one or more individual elements to perform the functions described herein.

Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A computer-implemented method, comprising:
receiving, by a machine learning model, a question associated with healthcare compliance from a user;
identifying, by the machine learning model, a healthcare compliance regulation document associated with the question and one or more healthcare compliance requirements corresponding to the healthcare compliance regulation document; and
recommending, by the machine learning model, a decision satisfying the one or more healthcare compliance requirements to the user.

2. The method of claim 1, further comprising:
identifying at least one precedent including a former decision made by another user associated with the question; and
recommending the decision satisfying the one or more healthcare compliance requirements based on the at least one precedent.

3. The method of claim 1, wherein recommending the decision comprises:
identifying at least one precedent including a former decision made by a former user associated with the question;
identifying at least one insight provided by another former user;
recommending a plurality of decisions to the user based on the at least one precedent and the at least one insight; and
receiving a selection among the plurality of decisions from the user.

4. The method of claim 1, further comprising:
receiving the healthcare compliance regulation document harvested from a health authority web site;
classifying the healthcare compliance regulation document into a first category;
identifying the one or more healthcare compliance requirements from the healthcare compliance regulation document;
classifying each healthcare compliance requirement into a second category;
determining that each healthcare compliance requirement is a new healthcare compliance requirement or an updated healthcare compliance requirement; and
in response to determination, storing the new healthcare compliance requirement or the updated healthcare compliance requirement in a database.

5. The method of claim 4, wherein the healthcare compliance regulation document is harvested from the health authority website by a robot.

6. The method of claim 4, further comprising:
receiving, from a subject-matter expert (SME), a first validation for classification of the healthcare compliance regulation document into the first category;
in response to the first validation, classifying the healthcare compliance regulation document into a third category;
receiving, from the SME, a second validation for each healthcare compliance requirement into the second category; and
in response to the second validation, classifying each healthcare compliance requirement into a fourth category.

7. The method of claim 4, further comprising:
extracting at least one healthcare compliance term from the healthcare compliance regulation document;
providing the at least one healthcare compliance term to a subject-matter expert (SME); and
adding the at least one healthcare compliance term to a healthcare compliance vocabulary engine upon approval of the SME.

8. The method of claim 1, further comprising training the machine learning model using the decision.

9. A computing system comprising:
one or more processors coupled to a memory, the processors and memory configure to perform the method of claim 1.

10. A non-transitory computer readable medium storing instructions for causing a computing system to perform the method of claim 1.

11. A computer-implemented method, comprising:
receiving a healthcare compliance regulation document harvested by a robot from a health authority web site;
classifying the healthcare compliance regulation document into a first category;
identifying one or more healthcare compliance requirements from the healthcare compliance regulation document;
classifying each healthcare compliance requirement into a second category;
determining that each healthcare compliance requirement is a new healthcare compliance requirement or an updated healthcare compliance requirement; and
in response to determination, storing the new healthcare compliance requirement or the updated healthcare compliance requirement in a database.

12. The method of claim 11, further comprising:
receiving, from a subject-matter expert (SME), a first validation for classification of the healthcare compliance regulation document into the first category;
in response to the first validation, classifying the healthcare compliance regulation document into a third category;
receiving, from the SME, a second validation for each healthcare compliance requirement into the second category; and
in response to the second validation, classifying each healthcare compliance requirement into a fourth category.

13. The method of claim 11, further comprising:
extracting at least one healthcare compliance term from the healthcare compliance regulation document;
providing the at least one healthcare compliance term to a subject-matter expert (SME); and
adding the at least one healthcare compliance term to a healthcare compliance vocabulary engine upon approval of the SME.

14. The method of claim 11, further comprising:
receiving a question associated with healthcare compliance from a user;
identifying a healthcare compliance regulation associated with the question and the one or more healthcare compliance requirements corresponding to the healthcare compliance regulation; and
recommending a decision satisfying the one or more healthcare compliance requirements to the user.

15. The method of claim 14, further comprising:
identifying at least one precedent including a former decision made by another user associated with the question; and
recommending the decision satisfying the one or more healthcare compliance requirements based on the at least one precedent.

16. The method of claim 14, wherein recommending the decision comprises:
identifying at least one precedent including a former decision made by a former user associated with the question;
identifying at least one insight provided by another former user;
recommending a plurality of decisions to the user based on the at least one precedent and the at least one insight; and
receiving a selection among the plurality of decisions from the user.

17. A computing system comprising:
one or more processors coupled to a memory, the processors and memory configure to perform the method of claim 11.

18. A non-transitory computer readable medium storing instructions for causing a computing system to perform the method of claim 11.
